(19) United States
(12) Patent Application Publication
(10) Pub. No.: US 2022/0409403 A1
(43) Pub. Date: Dec. 29, 2022

(54) STIMULATOR SUBSTRATE DEVICE AND DEVICE FOR STIMULATING NERVE CELLS

(71) Applicant: Saphenus Medical Technology GmbH, Krems an der Donau (AT)

(72) Inventors: Martin Brandstätter, Wien (AT); Bernhard Zach, Wien (AT); Andreas Mühlenberend, Weimar (DE); Aaron Pitschl, Wien (AT); Rainer Schultheis, Wien (AT)

(73) Assignee: Saphenus Medical Technology GmbH, Krems an der Donau (AT)

(21) Appl. No.: 17/771,663

(22) PCT Filed: Oct. 30, 2020
(86) PCT No.: PCT/IB2020/060227

§ 371 Date: Apr. 25, 2022

(30) Foreign Application Priority Data
Oct. 31, 2019 (EP) 19206619.9

(51) Int. Cl. A61F 2/68; A61B 5/103; A61B 5/11; A61B 5/00; A61N 1/04

(52) U.S. Cl. CPC A61F 2/68; A61B 5/1038; A61B 5/112; A61B 5/486; A61B 5/6802; A61N 1/0456; A61F 2002/6827

(57) ABSTRACT

A stimulator substrate device for stimulating nerve cells, and apparatus including same, in particular nerve cells in an extremity, includes a substrate support, in which a plurality of stimulators for emitting stimulation signals to the nerve cells are provided. The plurality of stimulators is provided in a line on the substrate support and the substrate support is tubular.

The invention relates to a stimulator substrate device according to claim 1 and a device for stimulating nerve cells according to claim 9.

On 8 Jun. 2015, in the Science Humanity section of the online daily newspaper derStandard.at, an article was published about the first prosthetic leg that feels sympathetically. The article explained in detail how severed nerves in a patient whose leg had been amputated were reactivated and relocated to a region of the skin on the stump of the leg, causing this area of skin to become particularly sensitive. The nerve endings were rerouted there in such manner that the former foot with six of its foot components, or the nerve area sections typical of the physiognomy these foot components, were mapped to six points in this area of the skin. In other words, a nerve area section typical of the physiognomy is a natural nerve area section that is relocated to the surface of an area of the skin which is actually not typical of it. Consequently, in this relocated foot, or the nerve area sections typical of the physiognomy, the patient felt not the skin (surface) of the leg stump which was normally therefore did not feel the skin (surface) of the leg stump there but as it were the elements of the sole of his foot, of his actually amputated leg.

Now in order to supply this sensed sole with real stimulation below a prosthetic foot—from its prosthetic sole—a total of six pressure sensors were placed in the heel region, the centre region, the forefoot region and the toe region. In these regions, one pressure sensor each was placed along the gait line of a human sole and one of the adapted artificial sole of a prosthetic leg, in six gait line-typical sole sections of the artificial sole. Each pressure sensor is connected to one nerve-stimulating stimulator/(actuator) via a data link and via a controller for the purpose of transmitting information to the corresponding six nerve area sections typical of the physiognomy. As is known from gait analysis, for example, a gait line at least extends from the lateral edge of the heel to the big toe of the sole. Accordingly, gait line-typical sole sections also extend from the edge of the heel to the toes and may be arranged parallel to the gait line, for example.

WO 2019/025838 A1 discloses a device which assigns at least two sensors, that is to say a first sensor and a second sensor also up to an n-th sensor to each gait line-typical sole section. These are connected to one stimulator each via a data connection and via a control. In this way, not just one information source, for example a pressure sensor, but at least two information sources are available for each sole section, and accordingly not just one digitally functioning stimulation source is available for each mapped sole section, but at least two whose mode of action is not digital but analogue. These stimulators can selectively stimulate the nerve area sections typical of the patient's physiognomy.

WO 03/086235 A2 discloses a device for monitoring a patient's state of imbalance. For this purpose, an array of sensors on the sole of the patient's own foot is used to measure whether the patient is in a balanced state. The sensor array is connected to an array of stimulators via a signal evaluator so that if a loss of balance occurs the imbalance signals generated by the signal evaluator are transmitted to the array of stimulators. The array of stimulators is disposed externally on a strap, and in use it is arranged on an extremity so that the patient is informed of the state of imbalance.

A disadvantage of this known solution is that when in use this plurality of stimulators must be arranged precisely and directly on an extremity of the patient in order to be able to transmit an imbalance signal to the patient's extremity that is strong enough to make the patient aware of an imbalance state at all.

In view of the situation as stated, the problem addressed by the present invention is thus to at least partially remedy the disadvantages of the related art, and in particular to create a universally usable stimulator substrate device having a plurality of stimulators for the reproducible transmission of stimulation signals as feedback signals to a user, and to create a universally usable device for the stimulation of nerve cells.

This problem is solved by the features of the independent claims. Advantageous developments thereof are presented in the figures and the dependent claims.

The stimulator substrate device according to the invention for stimulating nerve cells comprises a substrate support, in which a plurality of stimulators for emitting stimulation signals to the nerve cells are provided, wherein the plurality of stimulators is arranged along a line in the substrate support and the substrate support has a tubular structure.

Thus, when the stimulator substrate device is in use by the user, the array of stimulators is positioned at a distance from the user as it is arranged in the substrate support and/or inside the substrate support. In this way, its service life can be prolonged, since the plurality of stimulators can be protected from environmental influences such as dust, moisture, sweat, etc. In the present case, a tubular substrate support is understood to be a receptacle with at least one substrate support opening so that the plurality of stimulators can be protected circumferentially. In contrast to the related art described previously, this stimulator substrate device is not designed simply to capture or process measured signals for forwarding to a user via a feedback device. In a first information channel, this stimulator substrate device enables the reproducible transmission of stimulation signals to a user as feedback signals—regardless of the nature of the measurement signals captured and regardless of the purpose for which the measurement signals are evaluated. Consequently, this stimulator substrate device may be implemented in a wide range of applications and it can be used universally.

In particular, the stimulator substrate device functions to stimulate nerve cells at a user's extremity, so the device can function not only as a bionic prosthesis but also as a bionic orthotic. The stimulator substrate device may be implemented particularly for use as a bionic prosthesis, but also to assist patients/users with polyneuropathy or similar disorders, for sports medical purposes as well as for purely recreational purposes. Polyneuropathy often occurs in diabetic patients, and is characterized by a loss of feeling in the feet. As a result of the condition, pressure points lead to open wounds or sensitive areas on the feet which are difficult to treat.

The plurality of stimulators arranged along a line and in the substrate support are advantageously positioned behind one another or directly side by side. This enables the stimulator substrate device to be constructed in a compact and space-saving manner, so that it can be combined with a variety of items in common use such as a belt or a headband or the like.

In particular, the plurality of stimulators arranged in a line and in the substrate support are positioned in a straight line. Accordingly, the stimulator substrate device is constructed in a compact and space-saving manner, wherein the position of each of the plurality of stimulators can be moved and adjusted with precision.

More preferably, the substrate support surrounds at least a portion of the plurality of stimulators, so that at least a region of said substrate support performs the function of an encapsulation, so that they can be protected extremely effectively.

The substrate support is preferably flexibly movable. This enables the stimulator substrate device and the plurality of stimulators arranged in the substrate support to be applied easily and simply. The flexibly movable substrate support is made from a cloth material for example, so that it is convenient for use by the user.

The plurality of stimulators are preferably tactile stimulators. Tactile stimulators can be actuated easily and incrementally, and especially when they are connected to a controller. In this context, the stimulation frequency, the stimulation intensity, the duration of stimulation or the stimulation channel of the individual stimulators may in particular be controlled variously. Tactile stimulators are safe and practical, and at the same time powerful.

The plurality of stimulators are preferably vibrators. This enables conventional vibration generators, for example a Vibracall element to be used as stimulators for nerve cell ends. Historically, as an aspect of its intended use a Vibracall element typically provides for an emitted sound during the vibration process or produces a buzzing noise as part of the oscillation process. It is commonly known that Vibracall elements serve to cause the mobile phone device to vibrate as a whole, so that the owner can be made aware of an incoming call even when he does not hear a ringtone. In the same way, the user of the stimulator substrate device can also perceive a stimulation, optionally even without hearing a noise.

In particular, the plurality of stimulators each contain at least one eccentric element, by which the stimulation of the nerve cell ends can be effected simply. The respective eccentric element brings the respective stimulator into a vibrating state. The vibration is induced by the alternating centripetal force of the eccentric element. Since the centre of mass of the eccentric element is not located axis-centrally on the drive shaft of the stimulator, an imbalance is created which disturbs the smooth running of the drive and starts the stimulator vibrating. The farther the centre of mass of the eccentric element is from the axis of rotation, and the greater the mass and rotating speed are, the greater the centripetal force becomes and therewith also the amplitude of vibration of the stimulator.

Alternatively or additionally, at least some of the plurality of stimulators each include at least one oscillating element, by which the stimulation of the nerve cell ends can be effected in simple manner. The oscillating element brings the at least one stimulator into an oscillating state. The oscillation is generated by the oscillating element, wherein the respective oscillating element is accelerated via a short electromagnetic pulse and is then moved back to its original position again by the action of a spring mechanism. The mass moment of inertia generated thereby gives rise so a simple harmonic oscillation.

The plurality of stimulators preferably each have a drive motor for driving the respective eccentric element rotationally, so that the eccentric element may be caused to rotate.

Alternatively, the plurality of stimulators include a drive actuator for driving the oscillating element in oscillating manner, so that the respective oscillating element can be made to perform a harmonic oscillation.

More preferably, the substrate support is elastic. This enables the stimulator substrate device and the plurality of stimulators arranged in the substrate support to be placed on the user in a positionally secure and reproducible manner. The elastic substrate support is made for example from a rubber material, so that it can be returned to a dimensionally stable state after a deformation, and consequently the stimulator substrate device is seated firmly on the user's extremity or limb. The substrate support is preferably made from a fabric structure, which conforms easily to the user or his extremity. Thus, the substrate support is comparable to an item of clothing and is very comfortable to wear.

The plurality of stimulators are preferably made from a waterproof material. This renders at least the plurality of stimulators resistant to moisture, and in turn prolongs their useful life.

Alternatively or additionally, the substrate support is made from a waterproof material. In this way, undesirable absorption of moisture by the substrate support can be prevented, as this might cause the stimulators to shift their position in the substrate support.

Alternatively, the plurality of stimulators or the substrate support are coated with a waterproof material. This helps to prevent undesirable absorption of moisture by the plurality of stimulators or the substrate support, thereby further improving the range of uses of the stimulator substrate device.

The substrate support preferably includes a connecting section so that the energy (current/voltage) for the plurality of stimulators can be supplied to a section in the substrate support that is known and defined by the user. This has the effect of simplifying the handling of the stimulator substrate device for the user, so that he is also able to position the stimulator substrate device to best effect even in difficult situations.

The plurality of stimulators are preferably each connected to at least one connecting line, which in turn is connected to a controller for generating control commands for stimulation signals to drive the stimulators, wherein the at least one connecting line exits the substrate support in the area of the connecting section. The plurality of stimulators is actuated by the controller. The controller typically has a processor and a computing unit with several control programs including various control commands, by which the plurality of stimulators may be actuated systematically.

Alternatively or additionally, each of the plurality of stimulators is connected to at least one connecting line, which lines are connected to a supply device for driving the plurality of stimulators, wherein the at least one connecting line exits the substrate support close to the connecting section. The supply device supplies the energy (current/voltage) for the plurality of stimulators, thereby enabling the longest possible operating time of the stimulator substrate device.

An interface unit is preferably present for detaching the at least one connecting line from the controller and/or the supply device. An interface unit may be for example a plug connector, a clip connector or a bayonet lock, which enables a connection of the at least one connecting line to be detached from the controller and/or supply device.

The interface unit is advantageously connectable to various adapters, so that for example plugs conforming to various standards can be connected to the interface unit. For example, a 2-pin or a 3-pin or a multi-pin plug can be connected electrically to the adapter and the interface unit. This enables controllers with different control programs to be connected to the stimulator substrate device.

Advantageously, each of the plurality of stimulators is connected to a connecting line, each of which is connected in turn to the controller for generating control commands for stimulation signals to drive the respective stimulator. In this way, each of the plurality of stimulators can be actuated separately with control commands.

Advantageously, each of the plurality of stimulators is connected to a connecting line which is connected to the supply device for driving the stimulators. In this way, each of the plurality of stimulators can be supplied with energy separately.

Advantageously, the supply lines are bundled and arranged on the connecting section by means of a rigid supply line. The rigid supply line serves as kink protection, to prevent the supply lines from becoming bent in the area of the connecting section.

Each of the plurality of stimulators is advantageously equipped with a feedback unit for generating a feedback signal to the user. The feedback signal from the feedback unit shows the user (at least for training purposes), via another information channel, stimulation signals which are transmitted by the feedback unit to the plurality of stimulators. In particular, these feedback signals are acoustic or visual signals. Unlike the previous display methods, the intention is thus not necessarily to show the patient which signals are received at the sole, but which signals the stimulator is trying to emit to send and communicate to the user. This may be take place for example via Bluetooth®, WLAN, or in particular via mobile phone as the interface.

More preferably, the controller is equipped with a computing unit, wherein the computing unit may be designed to support various control programs, with which control modes may be called with different control commands, individually if necessary, depending on the respective control mode. In this way, the plurality of stimulators can be actuated with control commands according to at least one control program. Moreover, different movement processes, such as a running movement, a walking movement, a hill climbing movement etc. may be activated in the controller, and the control commands associated therewith may be transmitted to the plurality of stimulators and the user may be trained in this respect.

The controller is preferably designed to control the plurality of stimulators using at least one gait line-based sensor signal from at least one sensor in such manner that a stimulation typical of the physiognomy can be transmitted to the user's nerve cells, in particular the nerve cells of a physically existing extremity of the user. In this way, the stimulator substrate device may be used as a training apparatus and/or as a prosthesis and/or in a prosthesis. A gait line-based sensor signal, or a sequence of several gait line-based sensor signals are generated on the basis of information in the area of the gait line-typical sole sections of the human sole or an artificial sole by the sensor located there, in particular by a pressure sensor.

Alternatively or additionally, the controller includes a memory unit. Control programs with various control commands may be stored in the memory unit, and are thus easily retrievable by the controller.

The controller is preferably designed to control the plurality of stimulators using multiple gait line-based sensor signals from at least one sensor or from multiple sensors in such manner that a stimulation typical of the physiognomy may be transmitted to the user's nerve cells, in particular to the nerve cells of an extremity of the user.

The substrate support preferably has a mounting area, on which additional stimulators may be placed. This enables additional stimulators to be positioned in the substrate support, so that the stimulator substrate device can be adapted to individual needs of the user.

The additional stimulators are preferably of exactly the same design as the plurality of stimulators. Accordingly, they can be positioned in the mounting area simply and by the user himself, and maintenance of the stimulator substrate device can be carried out by the user himself.

In particular, the stimulators of the stimulator substrate device are connected to Augmented Reality devices to enable the user to be trained more effectively. For example, by means of a display device an avatar may suggest an ideal motion sequence for the user so that the needs of the user, such as training progress or an improved gait analysis, can be met quickly.

A device according to the invention for stimulating nerve cells comprising a stimulator substrate device such as is described herein and a fastening device for positioning the stimulator substrate device on the nerve cells of a user. In particular, the stimulator substrate device functions to stimulate nerve cells at an extremity of the user, to such effect that said device may serve not only as a bionic prosthesis, but also as a bionic orthotic. In particular, the stimulator substrate device may be implemented particularly for use as a bionic prosthesis, but also to assist patients/users with polyneuropathy or similar disorders, for sports medical purposes as well as for purely recreational purposes.

The fastening device is particularly a cuff or a prosthesis. A cuff allows flexible, elastic positioning of the stimulator substrate device. A prosthesis supports fixed and therefore reproducible positioning of the stimulator substrate device.

The stimulator substrate device is preferably positionable detachably on the fastening device. The stimulator substrate device may thus be separated from the device, for example, and connected electrically to a computer or tablet to back up simulation signal data which may be stored in the memory unit.

Alternatively, the stimulator substrate device is fixedly positionable on the fastening device, so that the stimulator substrate device is arranged immovably in the device, thereby ensuring that secure transmission of the stimulation signals can be guaranteed.

In particular, the stimulator substrate device is positionable on the fastening device in dimensionally stable manner. For example, the stimulator substrate device is bonded or sewn onto the fastening device, thus enabling a limited amount of flexibility despite its dimensional stability.

More preferably, the fastening device is elastic and/or flexible, which has the effect of further expanding the range of application of the device with the stimulator substrate device.

The fastening device is preferably made from another fabric structure, so that the substrate support of the stimulator substrate device described here can be sewn easily and so is mounted securely on the fastening device. For example, the additional fabric structure of the fastening device has a different fabric structure from the substrate support of the stimulator substrate device. Different fabric structures have different functional properties, so for example the fabric structure of the substrate support is designed to allow the plurality of stimulators to be arranged reproducibly on the extremity. For example, the additional fabric structure of the fastening device may be constructed elastically to simplify the arrangement of the device on the user.

In particular, the additional fabric structure is identical to the fabric structure of the substrate support of the stimulator substrate device. In this way, the device can be manufactured inexpensively.

The fastening device preferably has a mounting section for accommodating a controller for the stimulator substrate device. This enables the controller to be positioned close to the device in order to transmit the control commands reliably to multiple stimulators.

Additionally or alternatively thereto, the fastening device has a mounting section for accommodating a supply device for the stimulator substrate device. This enables the supply device to be positioned close to the device to enable an operationally reliable energy supply. The user thus has access to a section where he can arrange the supply device time after time.

The mounting section preferably has a mounting receptacle, in which the controller and/or the supply device can be arranged easily and detachably. User-friendly operation is enhanced thereby.

More preferably, a display device is provided, wherein the display device can be connected to the controller. The display device may be a mobile phone, in particular a smartphone, or a smart tablet sein, or it may also be arranged in the fastening device as a screen, wherein the fastening device comprises at least one touchscreen.

At least an item of information indicative for the stimulation signals of the plurality of stimulators may preferably be set using the display device. Indicative information is would include, but is not limited to the stimulation frequency, the stimulation intensity, the stimulation duration or the stimulation channel in order to adjust the respective plurality of stimulators. For example, maximum values or minimum values for the examples of indicative information given above may be set with the aid of the display device.

The display device is advantageously designed as a gait profile analyser in order to monitor physiognomically typical walking with the human foot or with an artificial foot, allowing a qualitative investigation of a motion sequence of the user, and delivering at least one associated feedback signal to the user. In this context, the gait profile analyser may be permitted to intervene in the emission of the feedback signals to influence the control of the motion sequence. The gait profile analyser may for example be integrated as an in a mobile phone as an application and/or may output an acoustic signal to the user.

The indicative information may advantageously be set in such manner that entire control programs can be created, and existing control programs can also be modified with the aid of the display device.

In particular, an acoustic signal and/or a visual signal that can be displayed on the display device or output from a loudspeaker of the device may be generated on the basis of the indicative information.

The fastening device is preferably a headband or a wristband or a glove or a belt or a garter, or a prosthesis shaft. This makes the stimulator substrate device extremely versatile and usable in a wide range of situations.

Further advantages, features and properties of the invention will be explained in the following description, in which exemplary embodiments of the invention are described with reference to the drawing.

The list of reference numerals is an integral part of the disclosure, as are the technical content of the patent claims and the figures. The figures are described in logical sequence and with regard to their cross-functionality. The same reference numerals denote identical components, reference numerals with different indices indicate functionally equivalent or similar components.

In the drawing:

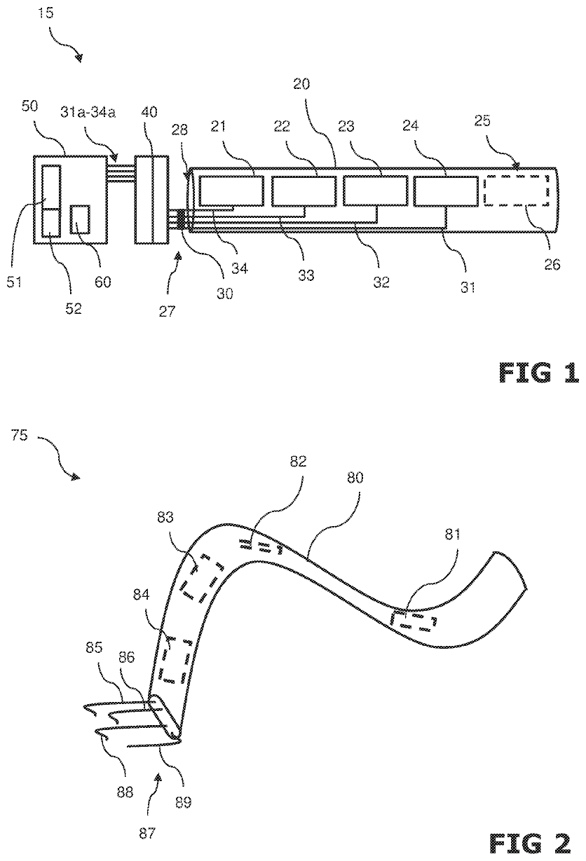

FIG. 1 is a perspective view of a first embodiment of a stimulator substrate device according to the invention,

FIG. 2 is a perspective view of a further embodiment of a stimulator substrate device according to the invention,

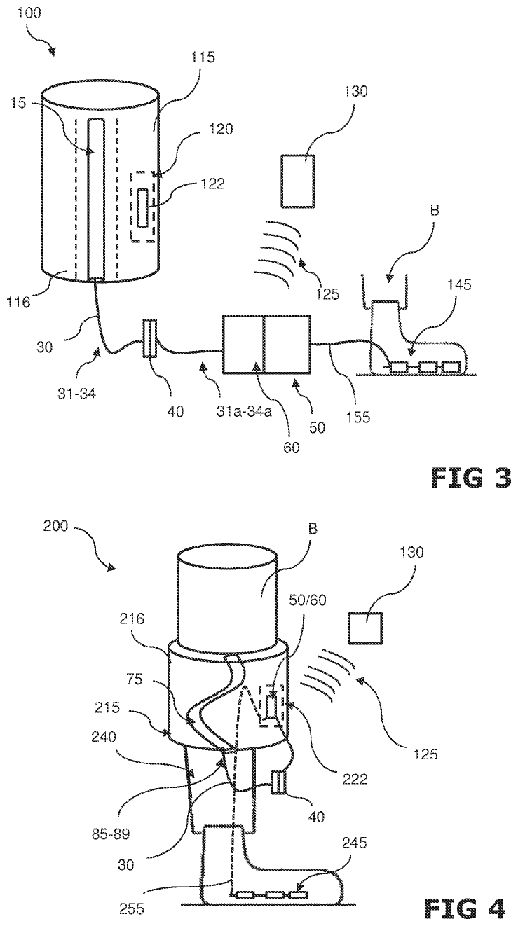

FIG. 3 is a perspective view of a first embodiment of a device according to the invention with a stimulator substrate device according to FIG. 1 for a human foot,

FIG. 4 is a perspective view of a second embodiment of the device according to the invention with a stimulator substrate device according to FIG. 2 for an artificial prosthesis,

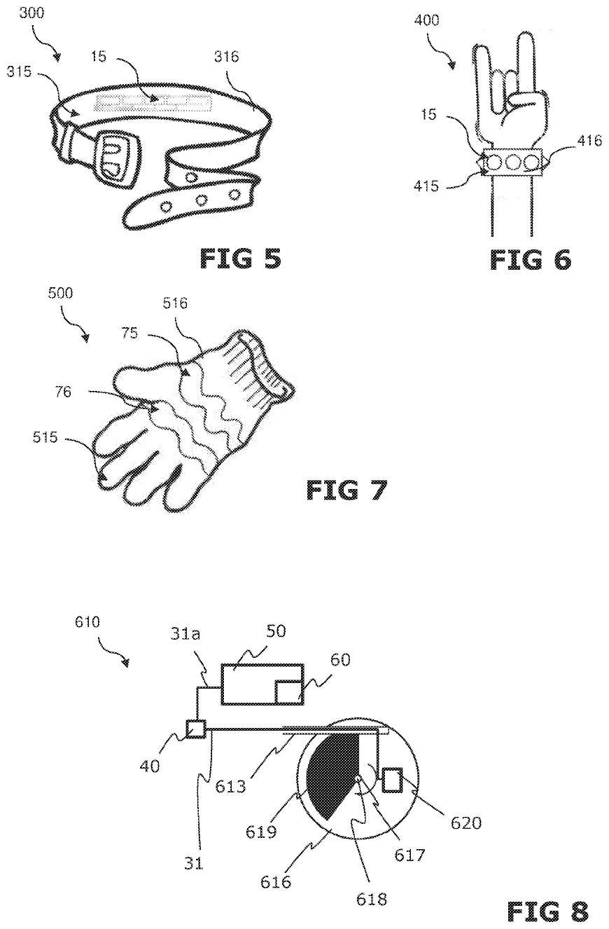

FIG. 5 is a perspective view of a further embodiment of the device according to the invention with a stimulator substrate device according to FIG. 1,

FIG. 6 is a perspective view of a further embodiment of the device according to the invention with a stimulator substrate device according to FIG. 1,

FIG. 7 is a perspective view of a further embodiment of the device according to the invention with a stimulator substrate device according to FIG. 2, and

FIG. 8 shows a stimulator for the stimulator substrate device according to FIG. 1 or according to FIG. 2.

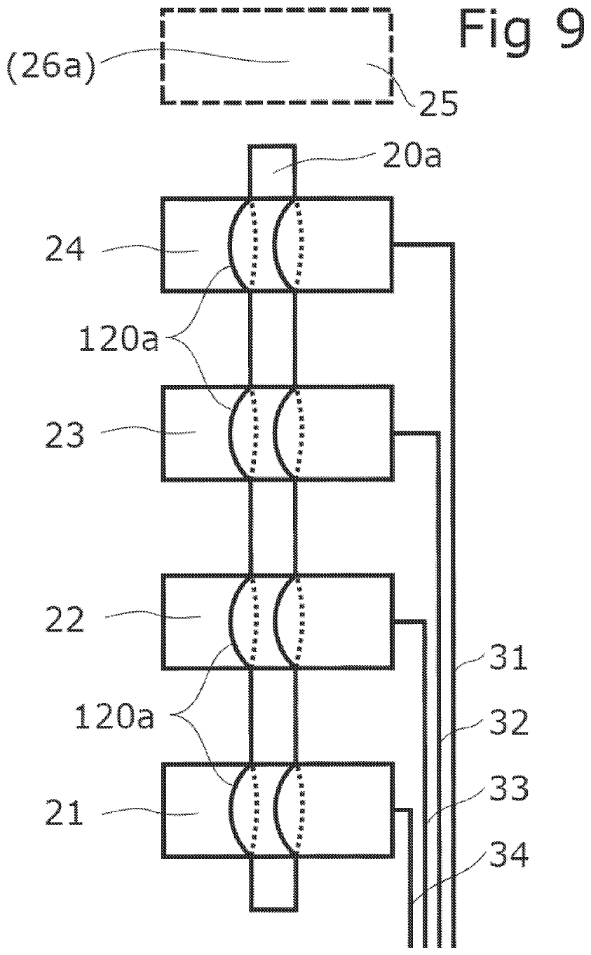

FIG. 9 shows a slightly modified arrangement of stimulators in a stimulator substrate device (arranged transversely to the lengthwise extension of the stimulator).

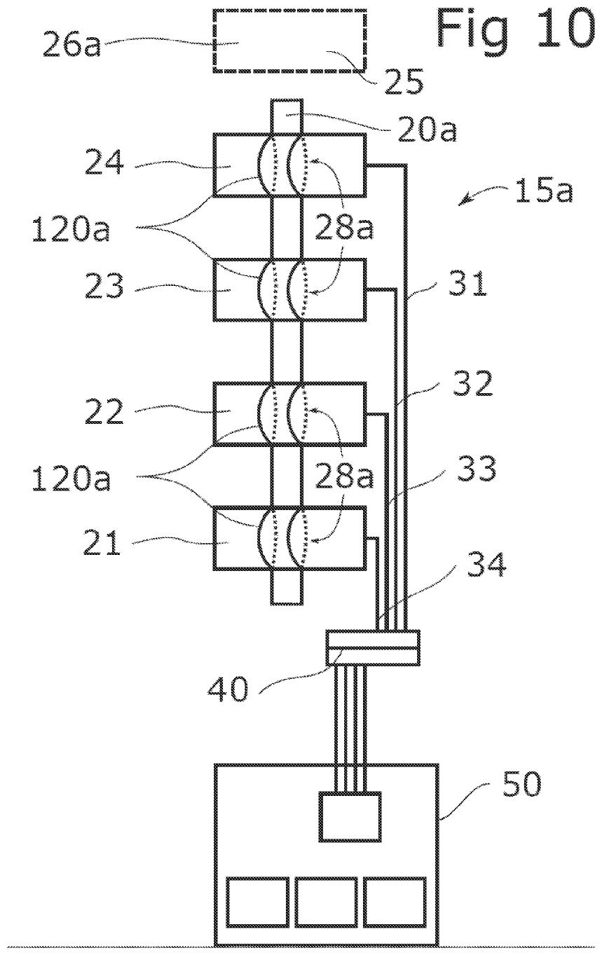

FIG. 10 shows the arrangement of FIG. 9 connected to the interface and the actuation device.

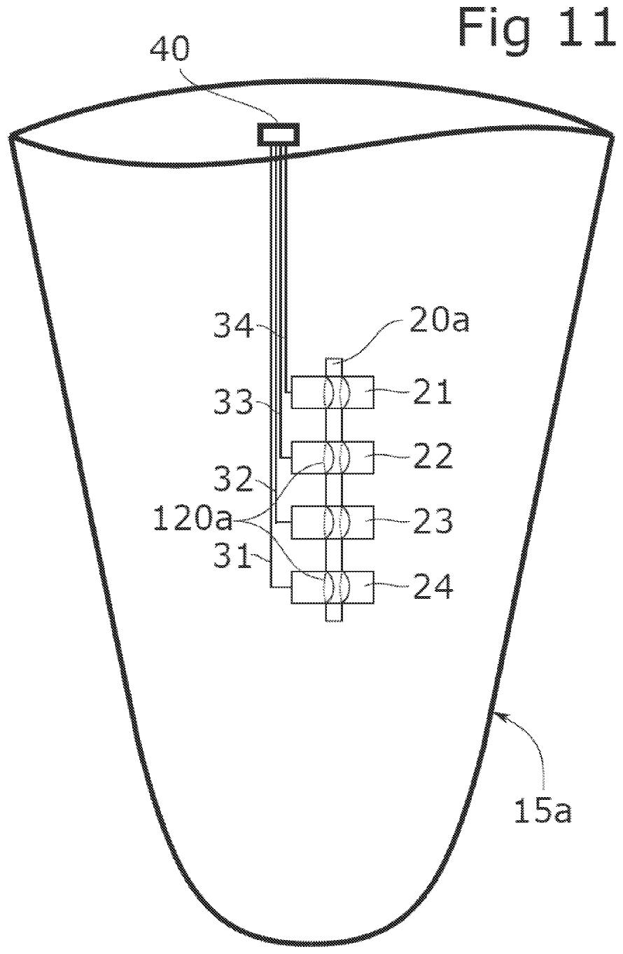

FIG. 11 shows the arrangement of FIG. 9 mounted in a liner for an amputation stump.

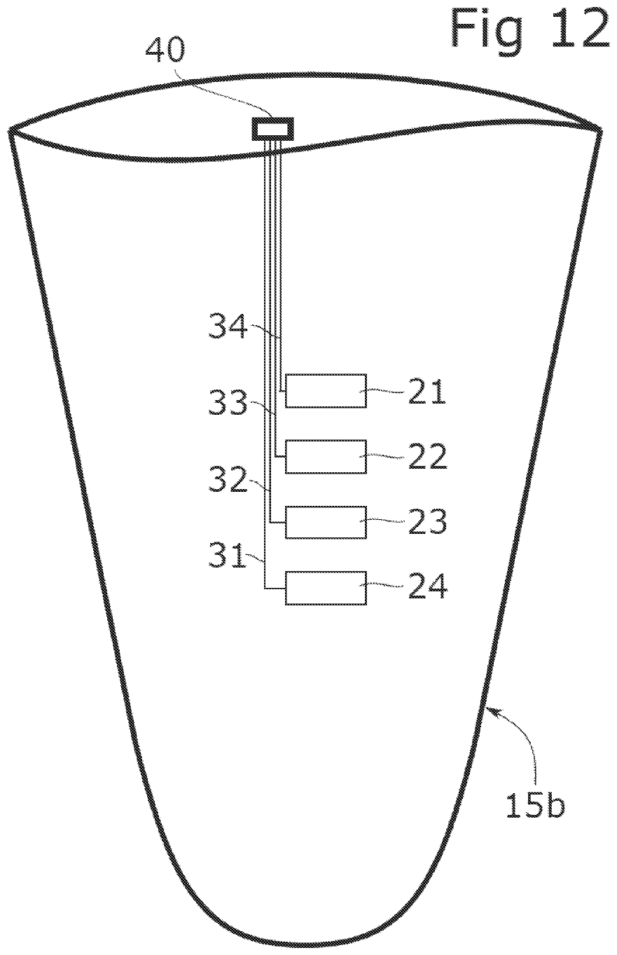

FIG. 12 shows a variant of the construction according to FIG. 11, wherein the stimulators are embedded directly in the liner material and as such are connected integrally/inseparably therewith without an intermediate carrier.

FIG. 1 shows a stimulator substrate device 15 for stimulating nerve cells on an area of the skin of a user B. The stimulator substrate device 15 comprises a substrate support 20, in which a plurality of stimulators 21, 22, 23, 24 are arranged for emitting stimulation signals to the nerve cells. The plurality of stimulators 21, 22, 23, 24 are arranged along a line and at a distance from each other in the substrate support 20, wherein the substrate support 20 is of tubular construction and consequently surrounds at least a portion of the stimulators 21, 22, 23, 24. The substrate support 20 is a receptacle which has at least one substrate support opening 28. The substrate support 20 further has a mounting area 26, on which further stimulators 25 may be positioned. These further stimulators 25 may be arranged in mounting area 26 of the substrate support 20 by user B according to his needs.

In the present instance, four stimulators 21, 22, 23, 24 are arranged on a line, positioned one behind the other or directly side by side in the substrate support 20, wherein the substrate support 20 is elastic and/or made from an elastic material, for example rubber or fabric. The substrate support 20 has a fabric structure which readily conforms to the user or an extremity of the user. The plurality of stimulators 21, 22, 23, 24 are coated with a waterproof material. This serves to prevent the plurality of stimulators 21, 22, 23, 24 from absorbing moisture.

Each of the plurality of stimulators 21, 22, 23, 24 is connected electrically with connecting lines 31, 32, 33, 34, which exit the substrate support 20 at a connecting section 27 of the substrate support 20. The supply lines 31, 32, 33, 34 are bundled and are arranged on the connecting section 27 with the aid of a rigid supply line 30. The connecting lines 31, 32, 33, 34 terminate in an interface unit 40 which is constructed as a detachable, 2-part plug connector. The interface unit 40 includes further connecting lines 31a, 32a, 33a, 34a, which connect the interface unit 40 electrically to a controller 50 and to a supply device 60 for driving the stimulators 21, 22, 23, 24, for example a battery or rechargeable battery. Various adapters may be arranged on the interface unit 40, so that for example plugs with different, country-specific standards—e.g., earthed connections—can be connected to the interface unit 40 (not shown). The controller 50 is designed to generate control commands for stimulation signals for driving the stimulators 21, 22, 23, 24. The controller 50 includes a computing unit 51 and a memory unit 52. The computing unit 51 is designed to support various control programs, with which control modes may be called with different control commands, individually if necessary, depending on the respective control mode. In this way, the plurality of stimulators may be actuated with control commands according to at least one control program. The control programs with various control commands are stored in the memory unit 52, so that they are retrievable by the controller 50.

FIG. 2 shows a stimulator substrate device 75 for stimulating nerve cells on an area of the skin of a user B in a further embodiment of the stimulator substrate device 15 according to FIG. 1. This stimulator substrate device 75 includes largely the same features and components as were described previously. The stimulator substrate device 75 includes a substrate support 80, in which a plurality of stimulators 81, 82, 83, 84 are positioned, wherein the plurality of stimulators 81, 82, 83, 84 are connected electrically with connecting lines 85, 86, 88, 89. The connecting lines 85, 86, 88, 89 exit the substrate support 80 at the connecting section 87 of the substrate support 80 and are electrically connected to the interface unit 40 of FIG. 1 described earlier. The substrate support 80 is flexibly movable. This allows the stimulator substrate device 75 and the plurality of stimulators 81, 82, 83, 84 arranged in the substrate support 80 to be attached easily to the user B. The flexibly movable substrate support 80 is made from a cloth material, for example, and includes a fabric structure which is made from a waterproof material. This serves to prevent undesirable moisture absorption by the substrate support 80.

FIG. 3 shows a first embodiment of a device 100 for stimulating nerve cells, having a stimulator substrate device 15 as described in FIG. 1. The device 100 is equipped with a fastening device 115 in the form of a flexible or elastic cuff 116 for positioning the stimulator substrate device 15 on the nerve cells of an area of the skin of an extremity of a user B, which may serve for example as a bionic orthotic on a human foot. Device 100 together with the stimulator substrate device 15 may be implemented for use as a bionic prosthesis, but also to assist user B with polyneuropathy or similar disorders, for sports medical purposes as well as for purely recreational purposes. The stimulator substrate device 15 is positioned detachably on the cuff 116 and is thus separable from the device 100. The cuff 116 consists of a further fabric structure, which is of the same construction as the fabric structure of the substrate support 20 of the stimulator substrate device 15. The fastening device 115 has a mounting section 120 for accommodating a controller 50 and has a supply device 60 for the stimulator substrate device 15. For this purpose, a mounting receptacle 122 in which the controller 50 and the supply device 60 may be accommodated is provided on the mounting section 120. The controller 50 is connected electrically via a sensor line 155 to sensors 145 for transmitting sensor signals, the sensors 145 being arranged on the sole of user B, or on a footwear or stocking worn by user B. In this context, the controller 50 is designed to control plurality of stimulators 21, 22, 23, 24 of the stimulator substrate device 15 by means of gait line-based sensor signals from the sensors 145, which have the form of pressure sensors for example, in such manner that a stimulation typical of the physiognomy may be transmitted to the nerve cells of user B, in particular to the nerve cells of an extremity of user B.

A display device 130 is also provided, wherein the display device 130 is connected to the controller 50 via radio signal 125. In the present case, the display device 130 is a smartphone equipped with a touchscreen.

The display device 130 may be used to set the information that is indicative for the stimulation signals of the plurality of stimulators 21, 22, 23, 24.

The stimulation frequency, simulation intensity, stimulation time or the stimulation channel for example may be changed by user B to adjust the respective plurality of stimulators 21, 22, 23, 24.

The display device 130 is embodied as a gait profile analyser for example, in order to monitor the action of walking with the human foot typical of the physiognomy, so that it allows a qualitative examination of a motion sequence performed by user B and gives user B at least one feedback signal in relation thereto. In this context, the gait profile analyser is permitted to intervene in the feedback signal transmission in order to influence the control of the motion sequence. The gait profile analyser is integrated in the smartphone as an application and outputs an acoustic signal to user B.

The plurality of stimulators 21, 22, 23, 24 of the stimulator substrate device 15 are connected to an Augmented Reality device which is integrated in the display device 130, in order to provide user B with improved training. For example, it may enable an avatar to suggest an ideal motion sequence for user B via a display device 130.

FIG. 4 shows a device 200 for stimulating nerve cells on an area of the skin of the extremity of a user B in a further embodiment of the device 100 according to FIG. 3. This device 200 has largely the same features and components as were described previously in device 100. Device 200 has a fastening device 215 in the form of a prosthesis shaft 216 of a prosthesis 240. The device 200 includes the stimulator substrate device 75 according to FIG. 2, which is positioned immovably on the fastening device 215. The controller 50 is electrically connected to the sensors 245 via a sensor line 255 for transmitting sensor signals, wherein the sensors 245 are arranged on the artificial foot or the prosthesis 240 and are in the form of pressure sensors. The sensor line 255 is routed inside the prosthesis 240. The plurality of stimulators 81, 82, 83, 84 of the stimulator substrate device 75 are connected electrically to the controller 50 via connecting lines 85, 86, 88, 89 as described previously. The controller 50 connected to the display device 130 via a wireless connection 125 for the exchange of control commands for the plurality of stimulators 81, 82, 83, 84. A mounting section with a mounting receptacle 222 in which the controller 50 and the supply device 60 may be accommodated is provided on the prosthesis shaft 216. The display device 130 is embodied as a gait profile analyser, for example, in order to monitor physiognomically typical walking with the artificial foot, allowing a qualitative investigation of a motion sequence of user B, and delivering at least one associated feedback signal to the user B. New control commands and control programs for the plurality of stimulators 81, 82, 83, 84 of the stimulator substrate device 75 may be created using the display device 130, and existing control programs may also be altered with the aid of the display device 130. For example, the display device 130 emits an acoustic signal and/or a visual signal which is displayed to user B via the display device 130, or is output by a loudspeaker of device 200. Each of the plurality of stimulators 81, 82, 83, 84 includes a feedback unit for generating a feedback signals for user B (not shown).

FIG. 5 shows a device 300 for stimulating nerve cells on an area of the skin of a user B in a further embodiment of the device 100 according to FIG. 3. This device 300 has largely the same features and components as were shown previously. Device 300 has stimulator substrate device 15 according to FIG. 1, wherein the fastening device 315 is a belt 316.

FIG. 6 shows a device 400 for stimulating nerve cells on an area of the skin of a user B in a further embodiment of the device 100 according to FIG. 3. This device 400 has largely the same features and components as were described previously. Device 400 is equipped with stimulator substrate device 15 according to FIG. 1, wherein the fastening device 415 is a wristband 416.

FIG. 7 shows a device 500 for stimulating nerve cells on an area of the skin of an extremity of a user B in a further embodiment of the device 200 according to FIG. 4. This device 500 has largely the same features and components as were described previously. Device 500 has a first stimulator substrate device 75 according to FIG. 2 and a further stimulator substrate device 76 with largely the same features and components as the stimulator substrate device 75 according to FIG. 2, wherein the fastening device 515 is a glove 516.

The stimulator substrate device 15 according to FIG. 1 and stimulator substrate device 75 according to FIG. 2 described here may also be arranged on a further fastening device in combination with one another. As well as the fastening devices shown, such a fastening device may also be a headband or a garter.

FIG. 8 shows one of the plurality of stimulators 21, 22, 23, 24 or 81, 82, 83, 84 of the stimulator substrate device 15 or 75 according to FIG. 1 or FIG. 2, wherein said stimulator is embodies as a vibrator 610. Vibrator 610 has a housing 616, which is arranged in the substrate support 20 or 80 with a fastening unit 613. An eccentric element 619 is arranged inside housing 616. The eccentric element 619 is arranged on the drive shaft 617 so as to be able to turn or rotate in the direction of the drive 618. The vibration of the vibrator 610 is caused by the eccentric element 619 as a result of the alternating centripetal force of the eccentric element 619. Since the centre of mass of the eccentric element 619 is not located axle-centrically on the drive shaft 617 of the vibrator 610, an imbalance is created which disturbs the smooth running of the eccentric element 619 (causes uneven running) and causes the vibrator 610 to start vibrating. The farther the centre of mass of the eccentric element 619 is from the axis of rotation of the drive shaft 617, and the greater the mass and rotating speed of the eccentric element 619, the greater the centripetal force becomes and therewith the amplitude of vibration of the vibrator 610. The housing 616 of vibrator 610 accommodates a drive motor 620 for driving the eccentric element 619 in rotary manner. The drive motor 620 is connected to the drive shaft 617 for this purpose. In the form shown, the vibrator 610 has the form of a button cell. The drive shaft 617, the eccentric element 619 and the drive motor 620 are all disposed inside the housing 616, which is constructed as a cylinder.

The drive motor 620 is electrically connected to the previously described interface unit 40 via the connecting line 31 and to the controller 50 and the supply device 60 via the connecting line 31a. In this arrangement, the stimulation frequency, simulation intensity, stimulation time or the stimulation channel of the vibrator 610 are controllable.

FIG. 9 shows a stimulator substrate device 15a which is equipped with stimulators 21, 22, 23, 24, optionally 25, for stimulating nerve cells on an area of the skin of a user. The stimulator substrate device 15a comprises a substrate support 20a in which plurality stimulators 21, 22, 23, 24 are arranged for sending stimulation signals to the nerve cells. The plurality of stimulators 21, 22, 23, 24, optionally 25, are arranged along a line and at a distance from each other in the substrate support 20a, wherein the substrate support 20 is of tubular construction inasmuch as a tubular lug 120a is provided for each stimulator and thus surrounds at least a portion of the stimulators 21, 22, 23, 24. Thus, the substrate support 20a is a receptacle having at least one substrate support opening 28a. The substrate support 20a further has a mounting area 26a, on which further stimulators 25 may be placed. These further stimulators 25 may be arranged in the mounting area 26 of the substrate support 20a by the user as needed.

In the present case, four stimulators 21, 22, 23, 24 inside the substrate support 20a are arranged along a line, positioned one behind the other or directly beside one another, wherein the substrate support 20a is elastic or made from an elastic material, for example from rubber or a cloth. The substrate support 20a has a durable, e.g., fabric structure, which conforms readily to the user or his extremity. As with the other embodiments, the plurality of stimulators 21, 22, 23, 24 are coated with a waterproof material or are embedded in a plastic (or rubber) liner together with their substrate support 20a in waterproof manner (see FIG. 11 or 12). In this way moisture absorption by the plurality of stimulators 21, 22, 23, 24 may be prevented.

In contrast to FIG. 1, the stimulators in FIGS. 9-12 are orientated transversely and include tubular lugs 120a. The advantage of this is that the lengthwise extension of the stimulator region is somewhat shorter. And if the stimulators are then connected fixedly to a liner, as shown in FIGS. 11 and 12, they offer minimal obstruction to the liner, which can be rolled up as before in order to be pulled over the stump of can be removed from a stump.

FIG. 10 shows the same construction as FIG. 9, wherein it is connected to an actuation device 50 via interface 40. In principle there is no change to the construction according to figures described above.

As mentioned earlier, FIG. 11 shows the integration and fastening of a substrate support directly on or in a liner of a patient's stump.

FIG. 12 advances a step further towards integration in that in this case the liner itself assumes the function of the substrate support 20a, and itself incorporates the stimulators 21-24 embedded in the correct position. This is what the stimulator substrate device in FIG. 12 is identified with numeral 15b, since in this regard it differs from the structures according to FIGS. 9-11. Of course, however, the construction according to FIG. 11 may also be completely embedded in the liner.

As is known the liner fulfils an important function in connecting the prosthesis to the stump, and its role becomes more critical still, as the invention provides that the stimulation signal transmission has now also been integrated therein.

Accordingly, since a liner is tubular per se, the embodiment according to FIG. 12 consequently falls within the scope of protection of claim 1.

Interface unit 40 and controller 50 correspond the components described earlier under these reference numerals.

### LIST OF REFERENCE NUMERALS

15 Stimulator substrate device
15a Stimulator substrate device with liner
15b Stimulator substrate device fully integrated in the liner
20 Substrate support
20a Substrate support for transverse stimulators
21 Stimulator
22 Stimulator
23 Stimulator
24 Stimulator
25 Stimulator
26 Mounting area
27 Connecting section
28 Substrate support opening
28a Transverse substrate support openings
29
30 Supply line
31 Connecting line
32 Connecting line
33 Connecting line
34 Connecting line
31a Additional connecting line
32a Additional Connecting line
33a Additional Connecting line
34a Additional Connecting line
40 Interface unit
50 Controller
51 Computing unit
52 Memory unit
60 Supply device
75 Additional stimulator substrate device
76 Additional stimulator substrate device
80 Substrate support
81 Stimulator
82 Stimulator
83 Stimulator
84 Stimulator
85 Connecting line
86 Connecting line
87 Connecting section
88 Connecting line
89 Connecting line
100 Device
115 Fastening device
116 Cuff
120 Mounting section
120a Tubular lug as mounting section
122 mounting receptacle
125 Radio signal
130 Display device
145 Sensors
155 Sensor line
200 Device
215 Fastening device
216 Prosthesis shaft
222 Mounting receptacle
240 Prosthesis
245 Sensors
255 Sensor line
300 Device
315 Fastening device
316 Belt
400 Device
415 Fastening device
416 Wristband
500 Device
515 Fastening device
516 Glove
610 Vibrator
613 Fastening unit
616 Housing
617 Drive shaft
618 Direction of drive
619 Eccentric element
620 Drive motor
B User

1.-31. (canceled)

32. A stimulator substrate device for stimulating nerve cells, comprising a substrate support, in which a plurality of stimulators are arranged for emitting stimulation signals to the nerve cells, wherein the plurality of stimulators is arranged along a line on the substrate support and the substrate support is of tubular construction.

33. The stimulator substrate device according to claim 32, wherein the substrate support includes a fabric structure or is made from a rubber material, such that it returns to a dimensionally stable state after a deformation.

34. The stimulator substrate device according to claim 32, wherein the substrate support at least partially encloses the plurality of stimulators and the substrate support is flexibly movable.

35. The stimulator substrate device according to claim 32, wherein the plurality of stimulators are tactile stimulators.

36. The stimulator substrate device according to claim 35, wherein the plurality of stimulators are vibrators and each has at least one eccentric element and/or an oscillation element.

37. The stimulator substrate device according to claim 32, wherein the substrate support is elastic.

38. The stimulator substrate device according to claim 32, wherein the plurality of stimulators and/or the substrate support includes a waterproof material or are coated with a waterproof material.

39. The stimulator substrate device according to claim 32, wherein:
the substrate support includes a connecting section, and the plurality of stimulators are each connected to at least one connecting line, which is connected to a controller for generating control commands for stimulation signals and/or a supply device for driving the stimulators,
the at least one connecting line exits the substrate support in the area of the connecting section, and
an interface unit is present for detachment of the at least one connecting line from the controller and/or the supply device.

40. The stimulator substrate device according to claim 39, wherein the controller is equipped with a computing unit and/or a memory unit and is designed to control the plurality of stimulators with the aid of at least one gait line-based sensor signal from at least one sensor in such manner that a stimulation typical of the physiognomy can be transmitted to the nerve cells of a user.

41. The stimulator substrate device according to claim 32, wherein the substrate support has a mounting area, on which further stimulators can be positioned.

42. The stimulator substrate device according to claim 32, wherein the substrate support has substrate support openings that extend transversely to the line to accommodate the stimulators, and/or that the substrate support is embedded in a liner, or that a liner incorporates the stimulators and thus also functions as the substrate support.

43. A device for stimulating nerve cells comprising:
a stimulator substrate device for stimulating nerve cells, comprising a substrate support, in which a plurality of stimulators are arranged for emitting stimulation signals to the nerve cells, wherein the plurality of stimulators is arranged along a line on the substrate support and the substrate support is of tubular construction; and
a fastening device for positioning the stimulator substrate device on the nerve cells of a user.

44. The device according to claim 43, wherein the fastening device is a cuff or a prosthesis.

45. The device according to claim 43, wherein the stimulator substrate device can be positioned detachably on the fastening device.

46. The device according to claim 43, wherein the stimulator substrate device can be positioned immovably on the fastening device.

47. The device according to claim 43, wherein the fastening device is elastic and/or flexible.

48. The device according to claim 47, wherein the fastening device includes a further fabric structure, wherein the further fabric structure is the same as the fabric structure of the substrate support of the stimulator substrate device.

49. The device according to any one of claim 43, wherein the fastening device has a mounting section for accommodating a controller and/or supply device for the stimulator substrate device.

50. The device according to claim 43, wherein a display device is present, wherein the display device can be connected to the controller and at least one item of information indicative for the stimulation signals of the plurality of stimulators, such as an acoustic signal and/or a visual signal, can be generated.

51. The device according to claim 43, wherein the fastening device is a headband or a wristband, or a glove, or a belt, or a garter, or a prosthesis shaft.
